(19) United States
(12) Patent Application Publication
(10) Pub. No.: US 2023/0026797 A1
(43) Pub. Date: Jan. 26, 2023

(54) APPARATUS FOR GENERATING A PLASMA AND METHOD FOR PERFORMING A PLASMA TREATMENT

(71) Applicants: TDK Electronics AG, München (DE); Nova Plasma Ltd., Kibbutz Megiddo (IL)

(72) Inventors: Amnon Lam, Kibbutz Giv'at Oz (IL); Eliezer Fuchs, Kibbutz Megiddo (IL); Betsalel Rechav, Nes Ziona (IL); Robert Krumphals, Deutschlandsberg (AT); Markus Puff, Graz (AT); Thomas Koidl, Graz-Puntigam (AT); Aviad Harhol, Ramat Hasharon (IL)

(21) Appl. No.: 17/792,733

(22) PCT Filed: Jan. 12, 2021
(86) PCT No.: PCT/EP2021/050477

§ 371 Date: Jul. 14, 2022

(30) Foreign Application Priority Data
Jan. 15, 2020 (DE) 10 2020 100 823.6

(51) Int. Cl. A61L 2/26; A61N 1/44; A61L 2/14; A61L 2/00; H05H 1/24

(52) U.S. Cl. CPC A61L 2/26; A61N 1/44; A61L 2/14; A61L 2/0011; H05H 1/2481; A61B 2090/049

(57) ABSTRACT

An apparatus for generating a plasma above a treatment object. The apparatus includes a plasma-generating element such that during operation the plasma is generated by the plasma-generating element. The apparatus further includes a sealing element attached to the plasma-generating element. The sealing element has a cavity and is configured to form a closed volume with a part of the treatment object, so that at least a part of the cavity is part of the closed volume and, during operation of the apparatus, the plasma is generated in the closed volume. The sealing element includes a reception region that is configured so that a part of the treatment object can be inserted into the reception region. A method is also disclosed for performing a plasma treatment to a treatment object using the apparatus.

Embodiments of the present invention are related to an apparatus for generating a plasma and to a method for performing a plasma treatment. In some embodiments, the apparatus may comprise a piezoelectric transformer as a plasma-generating element.

Piezoelectric transformers in plasma generating devices can be used to transform a low input voltage into a high output voltage. Due to the high electric fields caused by the high output voltage the surrounding gas can be ionized and thus a plasma can be generated. Also other applications with an electromagnetic electrode generating a high electric field are possible for generating a plasma. The plasma can be used, for example, for cleaning and/or modifying the surface of an object. For certain applications, it can be advantageous if the plasma generating device as well as the object to be treated are situated in a gas chamber so that the gas atmosphere and the gas pressure can be adjusted and optimized, since, for example, the ignition of a plasma can be facilitated in a reduced gas pressure. Furthermore, for instance in an atmospheric plasma ozone is produced. In a gas chamber the ozone can be contained and thus separated from the environment. Moreover, the ozone can be pumped from the gas chamber and be lead to a filter or a catalyst. However, for instance in connection with treatments of parts of the human body, the use of a gas chamber is usually not possible.

Prior art document DE 10 2013 113 941 A1 discloses an assembly for the treatment of wounds, wherein the assembly comprises a housing with a device for generating a plasma or an excited gas or gas mixture. By means of a fan in or on the housing an airflow is generated that emerges into an expansion element, which surrounds a wound area to be treated. In order to keep the pressure inside expansion element approximately at the pressure level of the environmental pressure, the expansion element has plural perforations through which excess reaction gas and reaction products can escape.

Prior art document DE 10 2018 209 735 A1 discloses a plasma device for treating body surfaces, comprising a main body that can be held in the hand, on which a plasma source is arranged, which is designed for generating a non-thermal plasma. The plasma device further comprises a spacer which is designed to define a distance between the plasma source and a body surface to be treated in the mounted state.

Prior art document DE 10 2013 107 448 A1 discloses an arrangement for germ reduction by means of a plasma, wherein the arrangement comprises a piezoelectric transformer and a dielectric film with a peripheral edge, which surrounds a region to be sterilized and forms a cavity in which plasma is ignited.

Prior art document DE 10 2017 105 401 A1 discloses a device for generating a non-thermal atmospheric pressure plasma. The device includes a piezoelectric transformer configured to ignite a non-thermal atmospheric pressure plasma in a process medium and a control circuit configured to apply an input voltage to the first piezoelectric transformer and to perform a modulation of the input voltage such that the first piezoelectric transformer generates an acoustic signal.

Prior art document WO 2018/167156 A1 discloses a plasma device, wherein the plasma generated by a piezoelectric transformer is guided by a plasma exit channel.

Objects of certain embodiments are to provide an apparatus for generating a plasma and a method for performing a plasma treatment.

These objects are, inter alia, achieved with the subject matters of the independent claims. Further embodiments and configurations are subject matter of the dependent claims.

According to at least one embodiment, an apparatus is described, which is an apparatus for generating a plasma above a treatment object. Furthermore, the apparatus is used in a method for performing a plasma treatment to a treatment object. The following description equally applies to the apparatus and to the method.

The treatment object can be any object that has a surface to which a plasma treatment is to be performed. In particular, the treatment object can be a part of the human body. For example, the treatment object can be a part of a finger or a toe including a finger nail or a toe nail, since a plasma treatment can for instance help to reduce a bacterial or fungal infection of a nail. Accordingly, the method for performing the plasma treatment to the treatment object can be a treatment of a nail to treat the nail and, preferably, to reduce a bacterial and/or fungal infection of the nail.

According to a further embodiment, the apparatus comprises a plasma-generating element, wherein, during operation of the apparatus, the plasma is generated by the plasma-generating element. In some embodiments, the apparatus comprises a plasma-generating element which comprises a piezoelectric transformer with a first region and a second region, extending between an end region in the first region and an end region in the second region. The first region can be an input region, while the second region can be an output region. The piezoelectric transformer is configured to and can be used to transform a low voltage at the first region to a high voltage at the second region, thereby ionizing the surrounding gas in the vicinity of the end region in the second region. Accordingly, during operation of the apparatus, the plasma is generated in the vicinity of the end region in the second region of the piezoelectric transformer. The end region in the second region can also be denoted as output-side end region. It is also possible that the embodiments described before and in the following are carried out with another plasma-generating element, for example a plasma-generating element having an electromagnetic electrode. The electrode can be associated with a corresponding electromagnetic transformer or another high-voltage generator. One or more features described in connection with the piezoelectric transformer can also apply to such plasma-generating element.

According to a further embodiment, the plasma-generating element comprises a housing. For example, the housing can be configured to support a piezoelectric transformer to form a plasma-generating element and to provide an electrical contacting to the first region of the piezoelectric transformer. In other words, the piezoelectric transformer is mounted in the housing. The housing can preferably enclose at least the first region of the piezoelectric transformer. The housing can have an opening so that at least the end region in the second region of the piezoelectric transformer is accessible. Alternatively, the housing can have a cap covering the end region in the second region of the piezoelectric transformer. In this case, the piezoelectric transformer can be completely enclosed by the housing. During operation, a primary plasma is then generated between the output-side end region and the cap and a secondary plasma is generated outside the cap as seen from the piezoelectric transformer.

Here and in the following, the term “secondary plasma” denotes the plasma that is in contact with the treatment object during the plasma treatment, i.e., the plasma that causes the desired effects of the plasma treatment. The term “primary plasma” can denote a plasma or also a mere arcing in the form of one or more electrical discharges that can occur between the plasma-generating element and the treatment object but that is separated from the treatment object by at least one component of the apparatus. Such component in the form of a layer or another form can be, for example, a cap of the housing as explained before and/or a metallic material and/or a dielectric material. It can also be possible that several primary plasmas are generated between the piezoelectric transformer and the treatment object. The sum of all such phenomena are denoted as the “primary plasma” herein. Generally, it can be advantageous to suppress the energy dissipation of a primary plasma, for instance by reducing gap sizes, in order to increase the energy transfer from the plasma-generating element to the plasma in contact with the treatment object namely to the secondary plasma.

In some embodiments, the end region in the second region is electrically associated to a remote electrode. In some embodiments, the end region may be electrically associated to the remote electrode via a conductor element. The end region in the second region can be for example connected directly to the conductor element. It can be also possible that the end region in the second region is covered by a cap and/or a conductive electrode, so that the cap and/or the conductive electrode can be directly connected to the conductor element and the end region in the second region is accordingly connected indirectly to the conductor element via the cap and/or the electrode. The conductor element, which can be a conductive wire or a conductive rod, can lead the high voltage at the end region in the second region or at the cap to the remote electrode. In some embodiments, the end region may be electrically associated to the remote electrode without Galvanic connection but by capacitive or inductive coupling or a combination of both. It is also possible in some embodiments that the remote electrode is electrically associated to a high-voltage generator other than a piezoelectric transformer.

According to a further embodiment, the apparatus comprises a sealing element. The sealing element comprises a cavity and is configured to form a closed volume with a part of the treatment object, so that at least a part of the cavity is part of the closed volume and, during operation of the apparatus, a plasma is generated in the closed volume. As explained in greater detail below, the sealing element can preferably have an opening configured to be shut or covered or sealed by a part of the treatment object.

According to a further embodiment, the sealing element is attached to the housing of the plasma-generating device. For example, the sealing element can be mounted on the housing or the housing can be mounted on the sealing element. In some embodiments, the housing with the piezoelectric transformer partly reaches into the cavity of the sealing element. In other words, a part of the housing can be located outside the cavity, while another part of the housing is located inside the cavity. For example, the end region in the second region of the piezoelectric transformer and/or an electrode associated to a high-voltage generator, is located inside the closed volume and, during operation of the apparatus, a plasma is generated in the closed volume. It can also be possible that the sealing element is attached to the housing via the conductor element described above, so that the remote electrode can be located at least partly in the cavity, while the piezoelectric transformer or another high-voltage generator can be located partly or completely outside the cavity.

In the method for performing the plasma treatment, the apparatus is placed on the treatment object so that the sealing element is arranged with the cavity on a surface of the treatment object or at least a part of the treatment object is inserted into a part of the cavity of the sealing element, so that the closed volume is formed by the sealing element and a part of the treatment object. Furthermore, the apparatus is operated to generate a plasma above a surface of the treatment object. Here and in the following, above can also mean over. If it is necessary to protect a part of the surface of the treatment object in the closed volume against the plasma during the plasma treatment, that part of the surface can be covered by a protection cover. The protection cover can comprise or be made of a dielectric material, for example in the form of a non-adhesive or adhesive foil or in the form of a deposited film such as a lacquer.

In contrast to placing the treatment object and a plasma-generating device in a gas chamber as described above, it is possible with the apparatus described here to create a closed volume, in which the plasma is generated and in which the part of the treatment object that is to be plasma-treated is placed. The closed volume is created by bringing together the apparatus, in particular the sealing element, and the treatment object. The term “closed volume” can in particular denote a volume that has only reduced or even inhibited gas exchange with the surrounding environment. In particular, gas escaping from the closed volume into the surrounding atmosphere can preferably be inhibited. Accordingly, the closed volume can be tightly sealed from the surrounding environment or can prevent at least 90% or at least 95% or at least 99% of the gas inside the closed volume from escaping into the surrounding environment during the duration of the treatment procedure. Alternatively or additionally, gas penetration from the surrounding environment into the closed volume can be limited to equal to or less than 10% or to equal to or less than 5% or even to equal to or less than 1% of the volume of the closed volume. In particular, an interface between the sealing element and the treatment object can be specifically configured so that the gas exchange is reduced or even inhibited. The term “closed volume” does, however, not exclude specific means for purposefully leading gas into the closed volume or purposefully removing gas from the closed volume, for instance by leading gas into the closed volume or pumping gas from the closed volume through at least one channel as explained further below. Additionally, it is also possible that gas from the surrounding atmosphere is sucked or drawn into the closed volume, preferably through the opening of the sealing element, and removed from the closed volume, preferably through a channel. Accordingly, there can be a gas flow from the surrounding atmosphere into and through the closed volume for example towards a pump and/or filter, which is described below in further detail. In case of such gas flow the pressure inside the closed volume can correspond, at least substantially, to the atmospheric pressure, i.e., the pressure of the surrounding atmosphere. The gas flow into the closed volume can prevent gas from escaping from the closed volume in an uncontrolled way into the surrounding atmosphere, so that the closed volume is closed to the surrounding atmosphere in the sense explained above.

In some embodiments, the apparatus can be a hand-held device, which prevents, even without the need of a gas chamber, an unwanted gas exchange between the region where the plasma is generated and the surrounding environment, so that for instance ozone, which can be harmful for human beings, can be significantly prevented from diffusing into the surrounding atmosphere.

According to a further embodiment, the sealing element at least partly has a cup-like or tube-like shape with an opening, a mounting part and a side part connecting the mounting part to the opening. In other words, the mounting part and the side part of the sealing element can at least partly form the cavity of the sealing element, which is accessible through the opening. If not placed on the treatment object, the plasma would be generated at least partly in the cavity of the sealing element and the ionized gas of the plasma could escape the cavity through the opening.

For example, a part of the plasma-generating device, for instance the piezoelectric transformer, reaches through the mounting part and the side part laterally surrounds a part of plasma-generating device. This can also mean that the housing reaches through the mounting part and the side part laterally surrounds a part of the housing. In case the plasma-generating device comprises a remote electrode, the remote electrode can be located in the cavity of the sealing element. In particular, the remote electrode can be located at the mounting part in the cavity of the sealing element.

According to a further embodiment, the sealing element is configured to be placed with the opening on a surface of the treatment object. In this case, the closed volume is separated from the environment by the interface, i.e., the area of contact, between the border of the sealing element surrounding the opening and the surface of the treatment object.

According to a further embodiment, the sealing element has a sealing lip surrounding the opening. The sealing lip can form the border of the sealing element and can thus be in contact with the treatment object when the apparatus is placed on the treatment object and can form a seal separating the closed volume from the surrounding environment. Preferably, the sealing lip comprises an elastic material, for instance an elastic plastic material such as a silicone and/or a polyurethane, so that particularly preferably the sealing lip can adapt to the surface of the treatment object, thereby effectively sealing the closed volume from the surrounding atmosphere. If the closed volume is held at a lower pressure than the surrounding atmosphere, the sealing lip can prevent a movement of the sealing element and thus of the apparatus over the surface of the treatment object, so that the apparatus can be held in place.

According to a further embodiment, the side part of the sealing element at least partly comprises an inelastic material, for instance an inelastic plastic material, metal, glass, ceramic or a combination thereof. Preferably, the side part can be at least partly stiff and withstand the forces that are generated when the closed volume is held at a lower pressure than the surrounding atmosphere, so that the end region of the piezoelectric transformer can be kept at a certain distance from the surface of the treatment object, so that preferably no part of a housing and/or any other part of the plasma-generating device, for instance the piezoelectric transformer or a remote electrode, can mechanically contact the treatment object. Consequently, by means of the sealing element the surface can be held at a distance from the output-side end region of the plasma-generating device, for instance the piezoelectric transformer, or from a cap covering the end region. The mounting part and the side part of the sealing element can comprise or be made of different materials. Preferably both the mounting part and the side part comprise or consist of an inelastic material, respectively. Alternatively, the mounting part and the side part can comprise or be made of the same material and can be formed in one piece.

Furthermore, it can also be possible that the side part comprises a partly elastic and partly inelastic bellows. The bellows can allow movement and/or tilting of the piezoelectric transformer relative to the surface of the treatment object even in case the sealing element is held in place for instance by a reduced pressure in the closed volume and, for example, has a sealing lip that is pressed against the surface. Preferably, the bellows can be configured not to collapse when the closed volume is held at a lower pressure than the surrounding atmosphere. For instance, the bellows can comprise stiff rings made of an inelastic material connected to each other by an elastic material.

According to a further embodiment, the sealing element comprises a reception region that is configured to receive a part of the treatment object, so that a part of the treatment object can be inserted through the opening into the reception region. The reception region can for instance be formed by a tube or a part thereof, which can be elastic or inelastic and which can be at least a part of the side part of the sealing element. Furthermore, the reception region can be at least partly formed by a memory foam.

Furthermore, the sealing element can comprise several components. For example, the components can be detachable. For performing the plasma treatment, the components can be assembled, thereby forming the sealing element.

For example, the sealing element comprises at least a first component and a second component, wherein the treatment object can be inserted in the first component and the first component is inserted in the second component. Accordingly, the first and second component can at least partly comprise the reception region. The first component can comprise the sealing lip. Furthermore, the first component can comprise a treatment opening so that the surface of the treatment object is accessible and not covered by material of the first component. The second component can cover the first component, so that, when the sealing element is assembled and the treatment object is inserted in the reception region, the first component is situated inside the second component.

At least one of the components of the sealing element, for example the second component, can form a dielectric barrier, so that, as explained above, during operation a primary plasma may be generated outside the cavity and a secondary plasma is generated inside the cavity.

Furthermore, the components of the sealing element can comprise a third component with a mounting part for mounting the plasma-generating device. Moreover, the third component can comprise a tube-like part for accommodating the first and second components. Preferably, for assembling the sealing element the second component is inserted in the third component and, in the assembled state, the third component can preferably be movable with respect to the second component. It can also be possible that the first and second component or the second and third component are formed as a single component.

Furthermore, the third component can comprise a conductive layer, for instance a metal film or layer, that is situated between the plasma-generating element and the second component. The conductive layer can form an electrode that allows transfer of energy from the plasma-generating element towards the treatment object, preferably through the second component as the only dielectric barrier.

Furthermore, the plasma-generating device can have a locking mechanism for detachably locking the plasma-generating device to the sealing element. For instance, the sealing element or a component thereof, for example the third component, can have a mounting part which forms a counterpart to the locking mechanism of the plasma-generating device.

According to a further embodiment, at least one of the housing and the sealing element has at least one channel reaching into the cavity of the sealing element. In particular, the at least one channel reaches from outside the cavity into the cavity through the housing or through the sealing element. By means of the at least one channel, the closed volume can be accessible for a pump or for a gas source, so that, during and/or before generating the plasma and performing the plasma treatment, the gas atmosphere and/or the gas pressure in the closed volume can be adjusted. Accordingly, the apparatus can further comprise a pump and at least one channel is connected to the pump. By means of the pump the pressure in the closed volume can be reduced. For example, a pressure of equal to or greater than 50 mbar and equal to or less than 600 mbar can be advantageous for a low-pressure operation of the apparatus. In addition to a pump, the apparatus can have a filter, in particular a filter for harmful gases, wherein the filter is connected to the channel. For instance, the filter can be a filter with a catalytic effect for decomposing ozone and/or other harmful gas components. Alternatively or additionally, the apparatus can further comprise a gas source and at least one channel is connected to the gas source. For example, the closed volume can be flooded with a gas that allows for a lower plasma ignition voltage than air. The gas source can comprise a gas tank and a valve, for instance a throttle valve, by means of which the gas is lead into the closed volume via the at least one channel. In case the gas in the closed volume shall have an atmospheric pressure or a pressure higher than the atmospheric pressure it can be possible that no pump is needed.

A hygienic protection layer may be provided to biologically isolate parts of the apparatus from the treatment object. A hygienic protection layer may be configured to envelope the part of the treatment object that is inside the closed volume so as to biologically isolate the sealing element from the treatment object. When the treatment object is, for example, a finger, the hygienic protection layer may be provided in a form of a glove or a thimble, possibly made of Latex or another flexible material, worn on the portion of the finger that is to be treated. Due to its flexibility, the hygienic protection layer may maintain similar pressure on both sides thereof while isolating the treated material from the sealing element as described above.

Further advantages, advantageous embodiments and further developments are revealed by the embodiments described below in connection with the figures, in which:

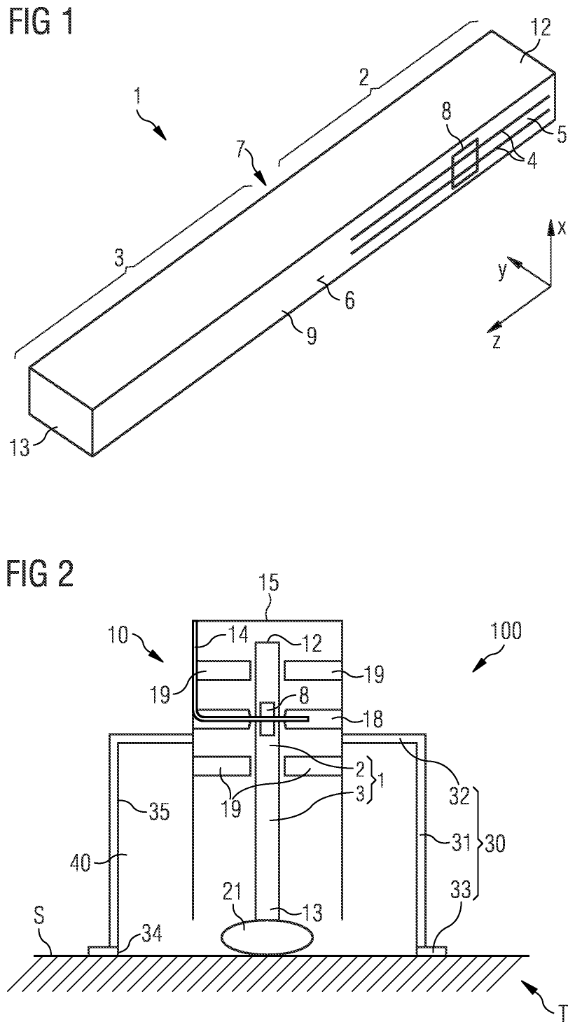

FIG. 1 shows a schematic illustration of a piezoelectric transformer according to an embodiment,

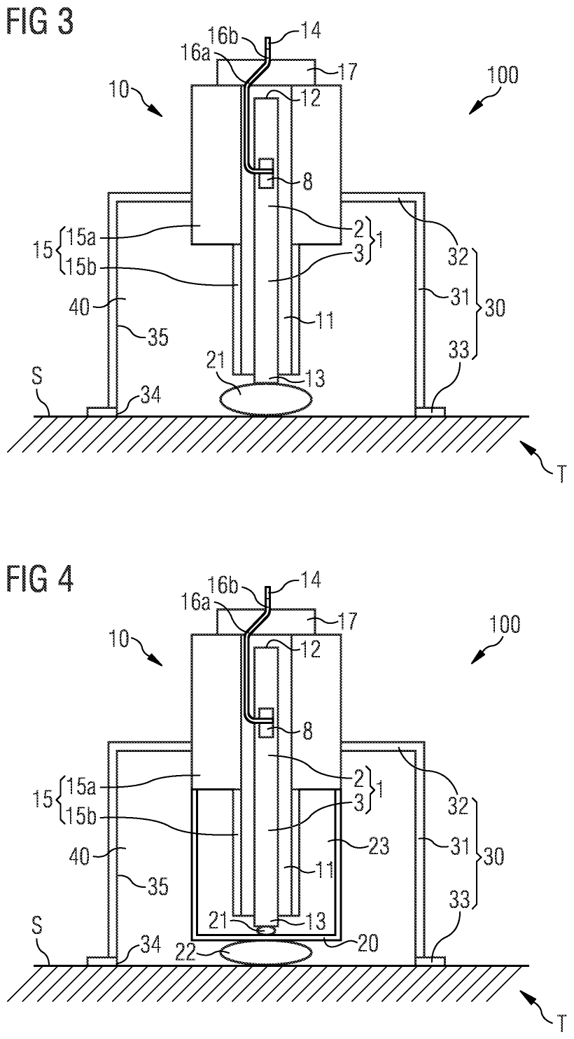

FIG. 2 shows a schematic illustration of an apparatus for generating a plasma for a method for performing a plasma treatment according to a further embodiment,

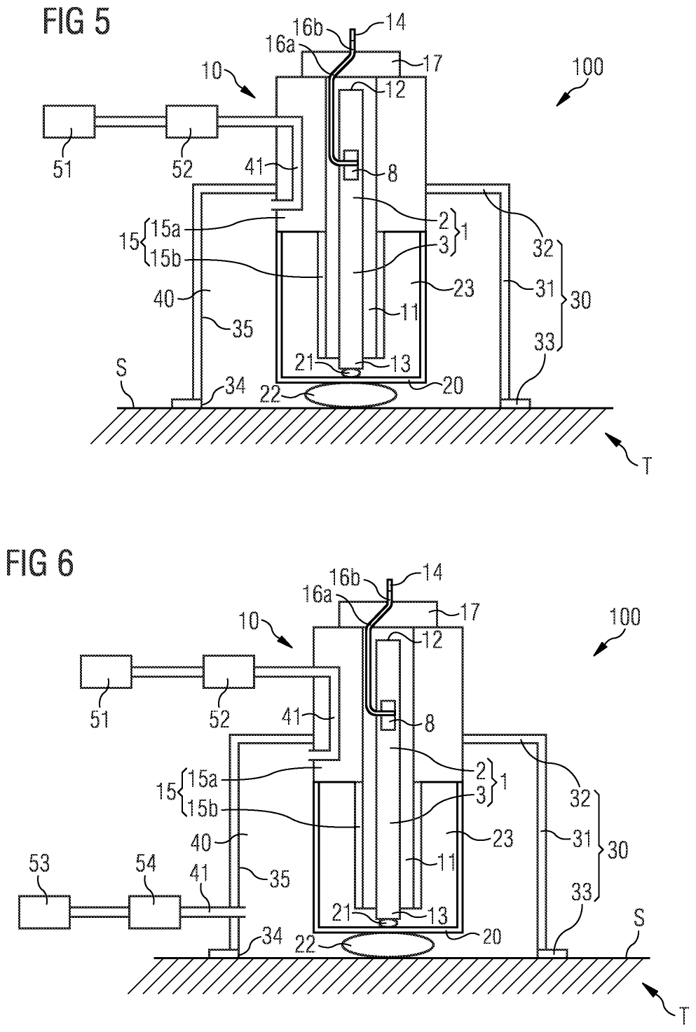

FIG. 3 shows a schematic illustration of an apparatus for generating a plasma for a method for performing a plasma treatment according to a further embodiment, and

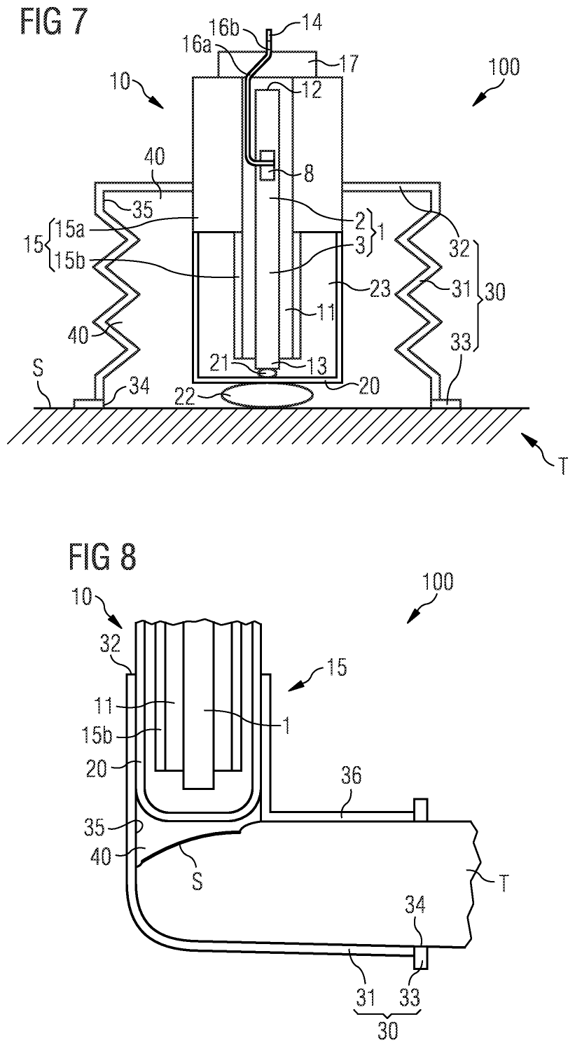

FIGS. 4 to 16K show schematic illustrations of an apparatus for generating a plasma for a method for performing a plasma treatment according to further embodiments.

In the embodiments and figures, identical, similar or identically acting elements are provided in each case with the same reference numerals. The elements illustrated and their size ratios to one another should not be regarded as being to scale, but rather individual elements, such as for example layers, components, devices and regions, may have been made exaggeratedly large to illustrate them better and/or to aid comprehension.

FIG. 1 shows, in a perspective view, an exemplary embodiment of a piezoelectric transformer 1 that can be a part of a plasma-generating element. In particular, the piezoelectric transformer 1 can be used as a part of a plasma-generating element in an apparatus for producing a plasma, in particular a non-thermal low pressure plasma or an atmospheric pressure plasma or a high pressure plasma, as shown in connection with the following embodiments. Preferably, the piezoelectric transformer 1 can be operated under reduced pressure or an atmosphere different from the ambient atmosphere. Although in the following embodiments a piezoelectric transformer is shown in connection with the plasma-generating device, other plasma-generating devices such as for example electrodes associated with corresponding electromagnetic transformers can be used.

A piezoelectric transformer 1 is an embodiment of a resonance transformer, which is based on piezoelectricity and, in contrast to conventional magnetic transformers, forms an electromechanical system. For example, the piezoelectric transformer 1 is a Rosen-type transformer. Alternatively, in the following embodiments other types of piezoelectric transformers can be used.

The piezoelectric transformer 1 has a first region 2 that is an input region and a second region 3 that is an output region, wherein the direction from the first region 2 to the second region 3 defines a longitudinal direction z. The first region 2 comprises an input-side end region 12 and the second region 3 comprises an output-side end region 13. In the first region 2, the piezoelectric transformer 1 comprises electrodes 4 to which an alternating voltage can be applied. The electrodes 4 extend in the longitudinal direction z of the piezoelectric transformer 1. The electrodes 4 are stacked alternately with a piezoelectric material 5 in a stacking direction x, which is perpendicular to the longitudinal direction z. The piezoelectric material 5 is polarized in the stacking direction x.

The electrodes 4 are arranged inside the piezoelectric transformer 1 between layers of piezoelectric material 5 and are also referred to as internal electrodes. The piezoelectric transformer 1 comprises a first side surface 6 and a second side surface 7, which is opposite the first side surface 6. On the first and second side surface 6, 7 external electrodes 8 are arranged. The internal electrodes 4 are alternately connected to one of the external electrodes 8.

The second region 3 comprises a piezoelectric material 9 and is free of internal electrodes. The piezoelectric material 9 in the second region 3 is polarized in the longitudinal direction z. The piezoelectric material 9 of the second region 3 can be the same material as the piezoelectric material 5 of the first region 2, but the piezoelectric materials 5 and 9 can differ in regard to their respective polarization direction. In particular, in the second region 3 the piezoelectric material 9 is formed into a single monolithic layer, which is completely polarized in the longitudinal direction z. The piezoelectric material 9 in the second region 3 has only one single polarization direction.

Via the external electrodes 8 a low alternating voltage can be applied between the electrodes 4 in the first region 2. Due to the piezoelectric effect of the piezoelectric material 5 the alternating voltage applied on the input side is converted into a mechanical oscillation. The frequency of the mechanical oscillation depends essentially on the geometry, the mechanical structure and the material of the piezoelectric transformer 1. Consequently, when an alternating voltage is applied to the electrodes 4 in the first region 2, a mechanical wave that generates an output voltage in the second region 3 by means of the piezoelectric effect is formed within the piezoelectric materials 5, 9. A high electrical voltage is generated between the output-side end region 13 and the electrodes 4 of the first region 2. This also creates a high potential difference between the output-side end region 13 and the surroundings of the piezoelectric transformer 1, sufficient to generate a strong electric field that ionizes a surrounding medium and causes the generation of a plasma. The field strength that is required for the ionization of the atoms or molecules or for the generation of radicals, excited molecules or atoms in the surrounding medium is referred to as the ignition field strength of the plasma. An ionization always occurs if the electric field strength on the surface of the piezoelectric transformer 1 exceeds the ignition field strength of the plasma.

FIG. 2 shows an embodiment of an apparatus 100 for generating a plasma 21 above a treatment object T used in a method for performing a plasma treatment to the treatment object T. The treatment object T can be any object that has a surface S which is to be plasma treated. For example, the treatment object T can be a part of a human body such as a finger or a toe or at least a part thereof. Alternatively, the treatment object T can be any object having a surface S comprising a material that for instance is to be cleaned and/or modified by a plasma treatment. In some embodiments, the apparatus 100 is a hand-held device that needs not to be placed inside a gas chamber together with the treatment object.

The apparatus 100 comprises a plasma-generating element 10 that comprises a piezoelectric transformer 1, which can be embodied as explained in connection with the foregoing embodiment. Alternatively, a plasma-generating device 10 with another type of piezoelectric transformer or, as mentioned above, a high-voltage electrode are possible.

In the shown embodiment, the piezoelectric transformer 1 is arranged in and supported by a housing 15, which provides the electrical contacting to the first region 2 of the piezoelectric transformer 1. The external electrodes 8 of the piezoelectric transformer 1 are electrically contacted by a wire connection 14 so that, during operation, the piezoelectric transformer 1 can be operated as explained above. Preferably, the wire connection 14 is soldered to the external electrodes 8. For controlling the piezoelectric transformer 1, the apparatus 100 can comprise control electronics which can be situated inside the housing 15 or outside the housing 15 in an additional electronics component (not shown).

The housing 15 is preferably at least configured to mechanically hold the piezoelectric transformer 1 in place. Furthermore, it can be advantageous if the housing 15 can also provide a mechanical support to the electrical connections to the piezoelectric transformer 1. As shown, the housing 15 can enclose at least the first region 2 of the piezoelectric transformer 1. In particular, the input-side end region 12 in the first region 2 is situated opposite an opening of the housing 15, through which at least the output-side end region 13 in the second region 3 of the piezoelectric transformer 1 is accessible. The housing 15 comprises support elements 18, which can support the piezoelectric transformer 1 preferably at a node point with regard to the oscillation of the piezoelectric transformer 1 during operation, which can be for instance at a length of one quarter of the total length of the piezoelectric transformer 1. The housing 15 can also have more support elements situated at various positions. Furthermore, as shown the housing 15 can comprise displacement protection elements 19. The displacement protection elements 19 can be spaced away from the piezoelectric transformer 1, when the piezoelectric transformer 1 is at a state of rest, i.e., not operated, and can form an end-stop against transverse movements of the piezoelectric transformer 1 during operation.

Furthermore, the apparatus 100 comprises a sealing element 30 that is attached to the housing 15 in the shown embodiment and that comprises a cavity 35. In particular, the sealing element 30 can be attached to the housing 15, for instance by adhering, soldering, brazing and/or mechanically fixing one of the housing 15 and the sealing element 30 to the other. As shown, the housing 15 with the piezoelectric transformer 1 can partly reach into the cavity 35 of the sealing element 30 so that a part of the housing 15 can be located outside the cavity 35 of the sealing element 30 and can be accessed there, while the other part of the housing 15 can be located inside the cavity 35. As explained in greater detail in the following, the sealing element 30 is configured to form a closed volume 40 with a part of the treatment object T, so that at least a part of the cavity 35 is part of the closed volume 40. Accordingly, the sealing element 30 has an opening 34 that is configured to be shut or covered or sealed by a part of the treatment object T. In the shown embodiment, the output-side end region 13 of the piezoelectric transformer 1 is located inside the closed volume 40 and, during operation of the apparatus 100, the plasma 21 is generated in the closed volume 40 as indicated in FIG. 2.

In order to conduct the method for performing the plasma treatment, the apparatus 100 can be placed on the treatment object T so that the sealing element 30 is arranged with the cavity 35 on the surface S of the treatment object T. It is also possible, as described further below, that at least a part of the treatment object T is inserted into a part of the cavity 35 of the sealing element 30. By either means, the closed volume 40 is formed by the sealing element 30 and a part of the treatment object T. When the closed volume 40 is formed, the apparatus 100 is operated to generate the plasma 21 above the surface S of the treatment object T.

As further shown in FIG. 2, the sealing element 30 can at least partly have a cup-like shape with a side part 31, a mounting part 32 and an opening 34, wherein in the shown embodiment the mounting part 32 is arranged opposite to the opening 34 and wherein the side part 31 connects the mounting part 32 to the opening 34. The side part 31 and the mounting part 32 form the cavity 35 of the sealing element, which is accessible through the opening 34. In the shown embodiment, the housing 15 reaches through the mounting part 32, while the side part 31 laterally surrounds a part of the housing 15 and the piezoelectric transformer 1.

The sealing element 30 is configured to be placed with the opening 34 on the surface S of the treatment object T. Depending on the sealing properties of the interface between the border of the sealing element 30 surrounding the opening 34 and the surface S of the treatment object, the closed volume 40 can be separated from the surrounding atmosphere. To that end, the sealing element 30 has a sealing lip 33 surrounding the opening 34 and forming the border of the sealing element 30. The sealing lip 33 is in contact with the surface S of the treatment object T when the apparatus 100 is placed on the treatment object T and forms the seal separating the closed volume 40 from the surrounding environment. As shown, the sealing lip 33 can preferably be formed like a rubber-ring and comprise an elastic material, for instance an elastic plastic material such as a silicone and/or a polyurethane, so that the sealing lip 33 can snugly fit to the surface S of the treatment object T. If the closed volume 40 is held at a lower pressure than the surrounding atmosphere, as described below, the sealing lip 33 can prevent a movement of the sealing element 30 and thus of the apparatus 100 over the surface S of the treatment object T, so that the apparatus 100 can be held in place.

The side part 31 of the sealing element 30 at least partly comprises an inelastic material, for instance an inelastic plastic material, metal, glass, ceramic or a combination thereof. Preferably, the mounting part 32 comprises also an inelastic material which can be the same as the material of the side part 31 or can be different. Accordingly, at least the side part 31 is at least partly stiff and can withstand the forces that are generated when the apparatus 100 is pressed onto the surface S and, as explained further below, the closed volume 40 is held at a lower pressure than the surrounding atmosphere, so that the output-side end region 13 of the piezoelectric transformer 1 can be kept at a certain distance from the surface S of the treatment object T. This can be advantageous in case that no part of the housing 15 and of the piezoelectric transformer 1 shall mechanically contact the treatment object T. The positions, geometries and dimensions of the piezoelectric transformer 1, the housing 15 and the sealing element 30 can be chosen so that the plasma 21 is generated close to the surface S of the treatment object T during operation of the apparatus 100.

FIG. 3 shows a further embodiment of the apparatus 100, which is a modification of the previous embodiment. In particular, the apparatus 100 of FIG. 3 comprises a plasma-generating element 10 with a modified housing 15 in connection with the piezoelectric transformer 1 and the sealing element 30 of the previous embodiment.

The piezoelectric transformer 1 is arranged in a potting compound 11 which encloses most of the side surfaces of the piezoelectric transformer 1. The input-side end region 12 of the piezoelectric transformer 1, which is arranged in the first region 2 and points away from the second region 3, is enclosed by the potting compound 11 or can alternatively protrude beyond the potting compound 11. The output-side end region 13 of the piezoelectric transformer 1, which is arranged in the second region 3 and points away from the first region 2, projects beyond the potting compound 11. Alternatively, the output-side end region 13 of the piezoelectric transformer 1 can also be covered by the potting compound 11. In addition, the wire connection 14, via which the piezoelectric transformer 1 is electrically contacted, is also partly covered by the potting compound 11. The potting compound 11 comprises a non-conductive material, which, preferably, is a soft, gel-like material. For example, the potting compound 11 can comprise silicone or consist of silicone. The potting compound 11 serves to prevent parasitic discharges on the side surfaces of the piezoelectric transformer 1.

The housing 15 surrounds the potting compound 11. In particular, the housing 15 has an opening which extends through the housing 15 and which is dimensioned in such a way that it accommodates the piezoelectric transformer 1 and the potting compound 11. As can be seen in FIG. 3, together with the potting compound 11 the piezoelectric transformer 1 is arranged in the housing 15 in such a way that the output-side end region 13 of the piezoelectric transformer 1 projects beyond the housing 15. Alternatively, both end regions 12, 13 can project beyond the housing 15 or the housing 15 can be flush with either one or both end regions 12, 13.

The housing 15 has a first housing section 15a and a second housing section 15b. The two housing sections 15a, 15b preferably comprise of different materials. The first housing section 15a encloses that part of the potting compound 11 which encloses the first region 2 of the piezoelectric transformer 1. The second housing section 15b encloses the other part of the potting compound 11 which surrounds the second region 3 of the piezoelectric transformer 1. In particular, the housing 15 can protect and mechanically stabilize the potting compound 11. By choosing different materials for the first and second housing sections 15a, 15b, the housing sections 15a, 15b can be well adapted to the different requirements in the first and second regions 2, 3 of the piezoelectric transformer 1.

The first housing section 15a preferably comprises a material with a high thermal conductivity, which can be a metal, a metal alloy, a thermally conductive plastic or a ceramic. For example, the first housing section 15a can comprise or consist of aluminum. In the first region 2 of the piezoelectric transformer 1 heat can be generated by ohmic losses and mechanical vibrations. Metal has a high thermal conductivity and is therefore well suited for dissipating the generated heat. In addition, metal is very robust, allowing for uncomplicated further processing and contacting of the first housing section 15a.

The second housing section 15b comprises or consists of an electrically non-conducting material. For example, the second housing section 15b can comprise or be made of plastic, Teflon, glass or ceramic. In the second region 3 of the piezoelectric transformer 1, high electric field strengths can occur. Since the second housing section 15b comprises or consists of an electrically non-conducting material, it does not influence the resulting electric field.

Furthermore, a cover 17 is arranged on the first housing section 15a. The cover 17 is preferably made of a material that is harder than the material of the potting compound 11 and can, for example, be made of an epoxy resin. As shown in FIG. 3, the wire connections 14 can be bent. For example, the wire connections can have two bends 16a, 16b above the potting compound 11. The cover 17 encloses in particular the two bends 16a, 16b of wire connections 14 so that the wire connections 14 are additionally mechanically fixed and a strain relief can be achieved, since tensile forces acting on the wire connections 14 can be absorbed in the cover 17 and do not act on the solder joints where the wire connections 14 are connected to the external electrodes 8 of the piezoelectric transformer 1.

As in the embodiment of FIG. 2, the sealing element 30 is attached to the housing 15 so that a part of the housing 15, in particular the second housing section 15b, is arranged inside the cavity 35 of the sealing element 30 and, thus, inside the closed volume 40 during a plasma treatment. As in the previous embodiment, the positions, geometries and dimensions of the piezoelectric transformer 1, the housing 15 and the sealing element 30 are chosen such that the plasma 21 is generated close to the surface S of the treatment object T during operation of the apparatus 100, while at least in some cases it can be preferable if no part of the housing 15 and of the piezoelectric transformer 1 mechanically contacts the treatment object T.

In contrast to the housings 15 of the embodiments of FIGS. 2 and 3, the housing 15 of the apparatus 100 of the embodiment shown in FIG. 4 additionally comprises a cap 20 to enclose the second area 3 of the piezoelectric transformer 1. The cap 20 can also be denoted as capsule. Inside the cap 20, air or a process gas different from air can be enclosed in a gas space 23. Inside the cap 20 a primary plasma 21 is generated at the output-side end region 13 of the piezoelectric transformer 1, while outside the cap 20 a secondary plasma 22 is generated due to electrical barrier discharges. In other words, the primary plasma 21 is generated between the output-side end region 13 of the piezoelectric transformer 1 and an inner wall of the cap 20 and the secondary plasma 22 is generated between an outer wall of the cap 20 and the surface S of the treatment object T.

Due to the cap 20, contamination of an area to be treated can be avoided. For example, it can be possible that during the plasma discharge material particles, which can contain Pb or another harmful material, can be detached from the piezoelectric transformer 1. The use of the cap 20 ensures that such particles do not get in contact with the surface S to be treated. In particular, if the apparatus 100 is used for medical or cosmetic purposes, such deposition must be avoided. Additionally, compared to a direct plasma ignition the use of dielectric barrier discharges has the advantage in medical or cosmetic applications that the tissue to be treated is less stressed.

The cap 20 can be made of an electrically non-conducting material, such as glass or aluminum oxide, in which case the cap acts as a dielectric barrier. In some embodiments the cap may be made of an electrically conducting material, such as stainless steel or aluminum. In this case, the cap 20 is electrically insulated against the first housing section 15a and can be maintained at a floating potential or alternatively can be grounded.

The gas space 23 can be permanently sealed and can be held a pre-determined pressure, which can be preferably lower than atmospheric pressure. In addition, the housing 15 can have a channel through which the gas space 23 can be pumped. For example, the pumped gas from the gas space 23 can be filtered by a filter before released to the environment. In some embodiments, the gas space 23 can be actively pumped or maintained at a predetermined pressure and gas composition through a system separated from a pumping system or gas system connected to the closed volume 40. Alternatively, the gas space 23 can have a fluid association with the closed volume 40, for instance via a channel through the cap 20, so that both the gas space 23 and the closed volume 40 can be pumped and evacuated together.

The gas space 23 can be filled with a process gas that can be, for example, a noble gas such as Ar, He or Ne. Alternatively or in addition, the gas space 23 can have a reduced pressure or even a vacuum. The ignition voltage can be reduced by reducing the pressure. The area of the primary plasma 21 is increased by the reduced pressure. Consequently, the area of the dielectric barrier discharge and, thus, of the secondary plasma 22 is also increased. The reduced pressure in the gas space 23 of the cap 20 can be used in combination with a reduced pressure in the closed volume 40 as explained in connection with the following embodiment.

Further embodiments of the apparatus 100 are shown in the following figures, which comprise, by way of example, the plasma-generating element 10 of the embodiment of FIG. 4. Alternatively, the plasma-generating element 10 and, in particular, the housing 15 for the piezoelectric transformer 1 can be embodied as described in connection with FIG. 2 or 3. Moreover, other plasma-generating elements are possible.

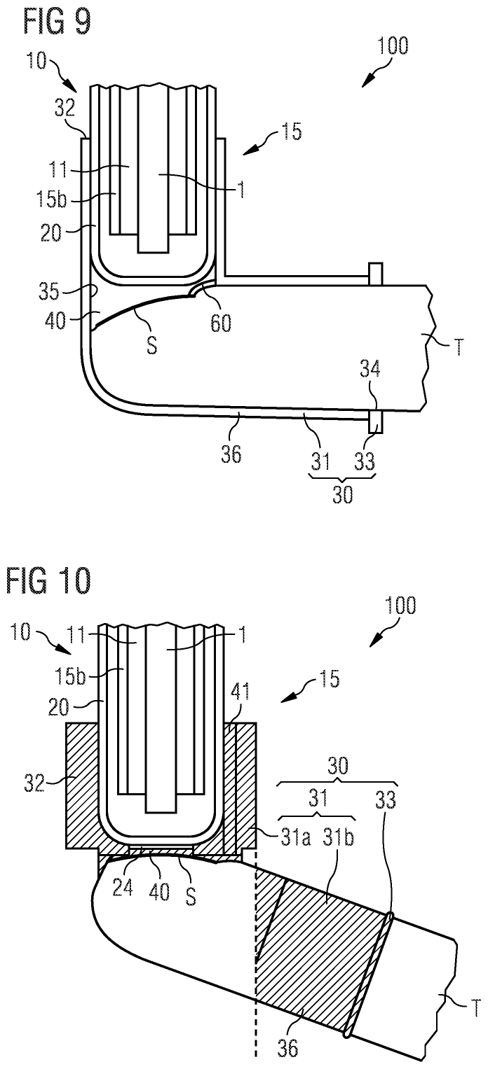

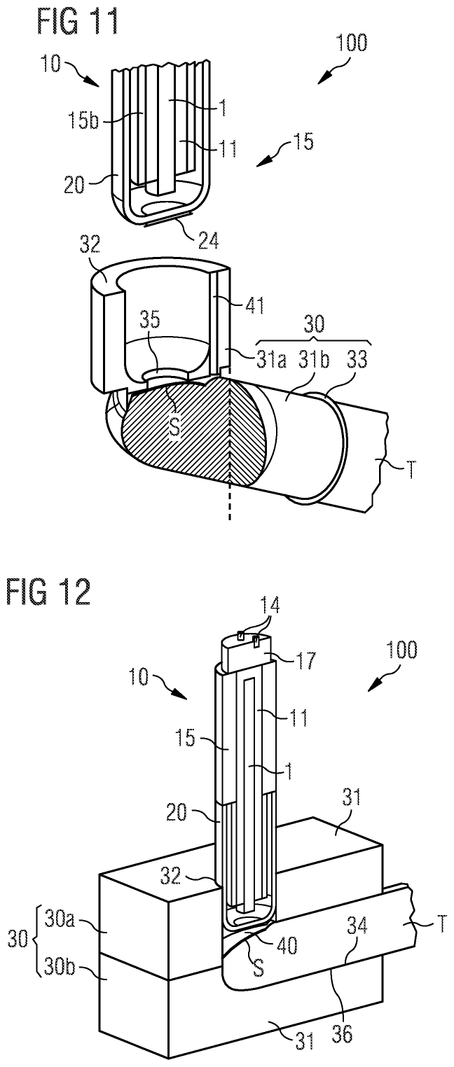

As shown in FIGS. 5 and 6, at least one of the housing 15 and the sealing element 30 can have at least one channel 41 reaching into the cavity 35 of the sealing element 30 and, thus, into the closed volume 40 during operation of the apparatus 100. In particular, the at least one channel 41 reaches from outside the cavity 35 into the cavity 35 through the housing 15 or through the sealing element 30.

According to the embodiment shown in FIG. 5, a pump 51 is connected to the cavity 35 via a channel 41 through the housing 15. Alternatively, the channel connected to the pump 51 can reach through the sealing element 30. By means of the pump 51 the pressure in the closed volume 40 can be reduced. For example, a pressure of equal to or greater than 50 mbar and equal to or less than 600 mbar can be advantageous for a low-pressure operation of the apparatus 100.

When reducing the pressure inside the closed volume 40 by at least partly evacuating the closed volume 40, ozone can be removed which can be generated during the plasma treatment. The apparatus 100 can have a filter 52 in addition to the pump 51, in particular a filter for harmful gases such as ozone, wherein the filter 52 is connected to the channel 41 and the closed volume 40 is pumped through the filter 52. For instance, the filter can be a filter with a catalytic effect for decomposing ozone. Consequently, the pump 51 can be protected from being damaged by the ozone. Furthermore, no ozone or only a small amount of ozone that is generated in the closed volume 40 during operation can reach the surrounding environment. This can be in particular advantageous in case of medical or cosmetic applications. Moreover, the ignition voltage of a plasma drops when the pressure is reduced. Accordingly, a plasma ignition is possible at lower operating voltages, as can be seen from the Paschen curve, and the region in which the plasma is generated is increased.

As shown in FIG. 6, alternatively or additionally the apparatus 100 can further comprise a gas source which is connected to the cavity 35 and, thus, to the closed volume 40 via a channel 41. For example, the closed volume 40 can be flooded with a gas that provides a lower plasma ignition voltage than air. The gas source can for example comprise a gas tank 53 and a valve 54, for instance a throttle valve, by means of which the gas is lead into the closed volume 40 via the at least one channel 41. In case the gas is intended to have an atmospheric pressure or a pressure higher than the atmospheric pressure, it can be possible that no pump is needed. It is also possible that only the housing 15 or only the sealing element 30 comprises channels 41.

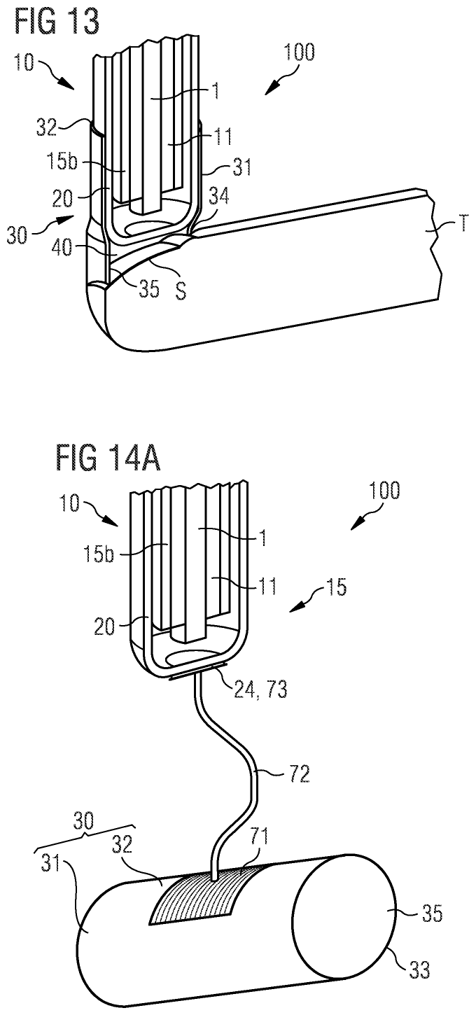

FIG. 7 shows a further embodiment of the apparatus 100, which comprises a sealing element 30 with a side part 31 that comprises a partly elastic and partly inelastic bellows. The bellows can allow movement and/or tilting of the piezoelectric transformer 1 and the housing 15 and thus of the generated plasma relative to the surface S of the treatment object T even in case the sealing element 30 is pressed against the surface S and held in place for example due to a reduced pressure in the closed volume 40. Accordingly, the treatment area can be increased. The bellows can be configured so as not to collapse when the closed volume 40 is held at a lower pressure than the surrounding atmosphere. For instance, the bellows can comprise stiff rings made of an inelastic material that are connected by an elastic material.

The following figures show embodiments of the apparatus 100, which are particularly advantageous for treating a finger nail or toe nail by applying a plasma. For example, the treatment object T can be a part of a finger or a toe including a finger nail or a toe nail, since it could be shown in studies that a plasma treatment can help to reduce a fungus infection of a nail. It can be particularly advantageous if such treatment is performed in a gas atmosphere with a reduced pressure. However, the features and components of the apparatus 100 described in the following are not limited to applications regarding plasma treatments of nails.

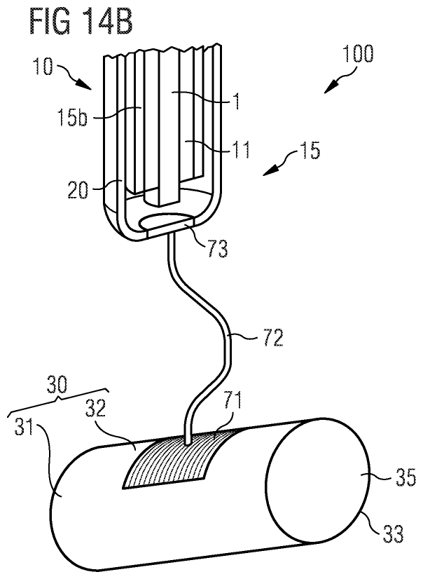

In contrast to the previous embodiments, the apparatus 100 of the embodiment shown in FIGS. 8 to 13 comprises a sealing element 30 having a reception region 36 that is configured such that a part of the treatment object T, for instance a part of a finger or a toe, can be inserted into the reception region 36 which is a part of the cavity 35. The reception region 36 can for instance be formed by a tube or a part thereof, which can be elastic or inelastic and which is at least a part of the side part 31 of the sealing element 30.

As shown in FIG. 8, the sealing element 30 is formed as an at least partly flexible tube which is configured to seal the closed volume 40 against the surrounding atmosphere. The sealing element 30 has a mounting part 32 that is attached to the cap 20 of the housing 15 in which the piezoelectric transformer 1 is arranged. The reception region 36 encloses a part of the treatment object T. As described in connection with the foregoing embodiment, the opening 34, through which the treatment object T can be inserted into the cavity 35 of the sealing element 30, can be surrounded by a sealing lip 33, which can be, for instance, a rubber ring. The pressure in the closed volume 40 can be reduced by pumping as explained before, for instance through a channel (not shown) in the sealing element 30. The side part 31 of the sealing element 30 is at least so stiff that the side part 31 is not pulled into the closed volume 40. This can be achieved, for instance, by stiff rings, which are pressure-stable, in the side part 31 of the sealing element 30.

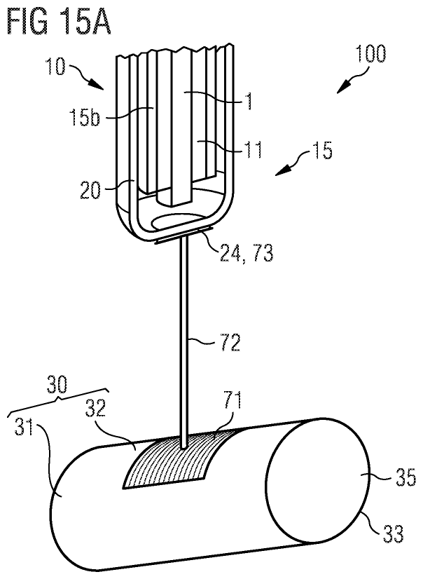

If it is necessary to protect a part of the surface S of the treatment object T in the closed volume 40 against the plasma during the plasma treatment, that part of the surface S can be covered by a protection cover 60 as shown in FIG. 9. The protection cover 60 can comprise or be made of a dielectric material, for example in the form of a non-adhesive or adhesive foil or in the form of a deposited film on the treatment surface S such as a lacquer. For instance in medical applications, the different electrical conductivities for example of a nail and the surrounding tissue can cause unwanted effects, since plasma more easily ignites close to a material with higher electrical conductivity and the surrounding tissue has a higher electrical conductivity than the nail. To avoid this effect, the area to be protected can be covered with the protection cover 60.

A hygienic protection layer may also be provided in some cases, to isolate parts of the apparatus 100 from the treatment object. For example, it may be advantageous to isolate the sealing element 30 from an infected finger that is treated by the apparatus, so that biological (or other) contamination cannot be transferred from the finger to the sealing element or vice versa. A hygienic protection layer may be provided for example by a flexible layer such as Latex that covers, envelopes or encapsulates the entire section of the treatment object that is inside the closed volume 40. In some embodiments, a Latex glove or a Latex thimble that is worn on the treated finger may serve as a hygienic protection. Such a flexible hygienic protection layer may be particularly advantageous because, while being biologically sealed, it allows for maintaining similar pressure on both sides of the layer. In other words, when an infected finger is introduced into the closed volume, thereby closing (sealing) the sealing element, and the closed volume is pumped, a pressure difference on both sides of the flexible layer may cause the portion of the layer inside the closed volume to swell, thereby maintaining a reduced pressure adjacently to the finger. The reduced pressure adjacent to the finger facilitates, in turn, plasma ignition adjacent to the finger. Such a hygienic protection may be a disposable item that is intended for a single use. Apart from being a biologic insulator, the flexible hygienic protection layer may also serve as a dielectric for the plasma as well. In this regard, it may serve as an additional dielectric or even the only dielectric in the closed volume.

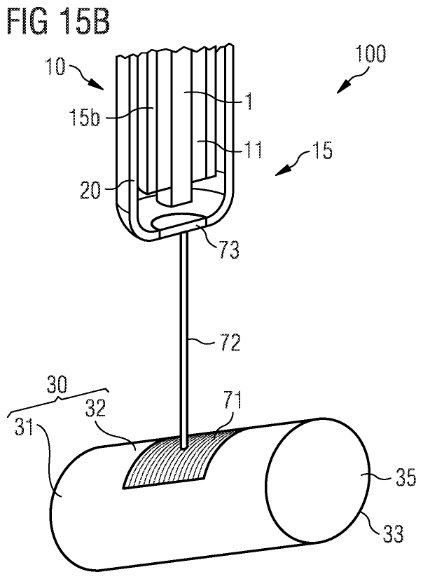

In order to ensure a uniform treatment result, it can be advantageous if the distance to the surface S for the ignition of the plasma is as uniform as possible. As shown in FIG. 10 in a partly sectional view, this can be achieved by forming a part 31a of the side part 31 as a spacer made of an inelastic material that can receive a part of the housing 15 for the piezoelectric transformer 1. The part 31a can also have a channel 41 reaching into the closed volume 40. The reception region 36 can be formed by the other part 31b of the side part 31 of the sealing element 30 and can be embodied as described above. By applying an electrically conductive layer 24 to the cap 20, it is possible to bundle the plasma discharge into the closed volume 40. The electrically conductive layer 24 can be, for example, a metal film.

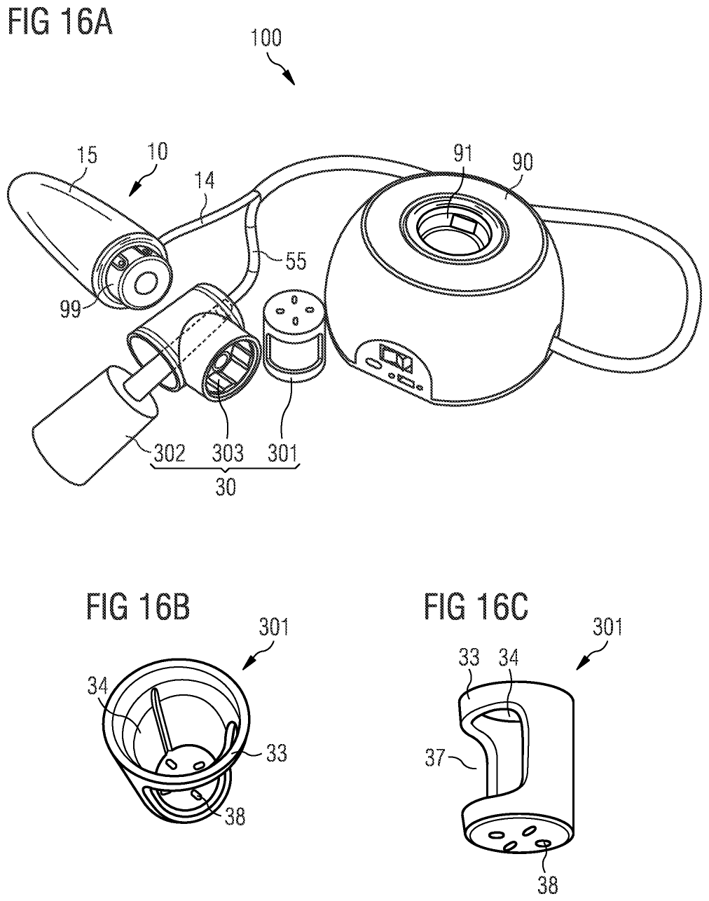

As shown in FIG. 11, it can also be advantageous if the plasma-generating device 10, i.e., in the shown embodiment the housing 15 with the piezoelectric transformer 1, and the sealing element 30 can be separately provided to the treatment object T. First the sealing element 30 can be arranged on the treatment object T. In case of the embodiment shown in FIG. 10 this can mean that the finger or toe to be treated is arranged in the reception region 36 of the sealing element 30. Afterwards, the housing 15 with the piezoelectric transformer 1 is arranged on the part 31a of the sealing element 30 that is formed as spacer as described before. In order to seal the cavity 35 from the surrounding atmosphere, it can be possible that the mounting part 32 comprises a covering part, for instance a thin material layer, covering the cavity 35, so that, independent from the position of the plasma-generating element 10, no additional sealing means are necessary to seal the closed volume from the surrounding atmosphere. In this case, the sealing element 30 forms together with the treatment object T a vacuum chamber that is separated from the plasma-generating element 10, so that even a layer of ambient air can be arranged between the plasma-generating element 10 and the mounting part 32. As shown, a channel 41 can reach into the cavity 35. It can also be possible that a sealing ring or other sealing means are arranged between the housing 15 and the mounting part 32 so that the closed volume is sealed from the surrounding atmosphere when the plasma-generating element 10 is placed in the mounting part 32.

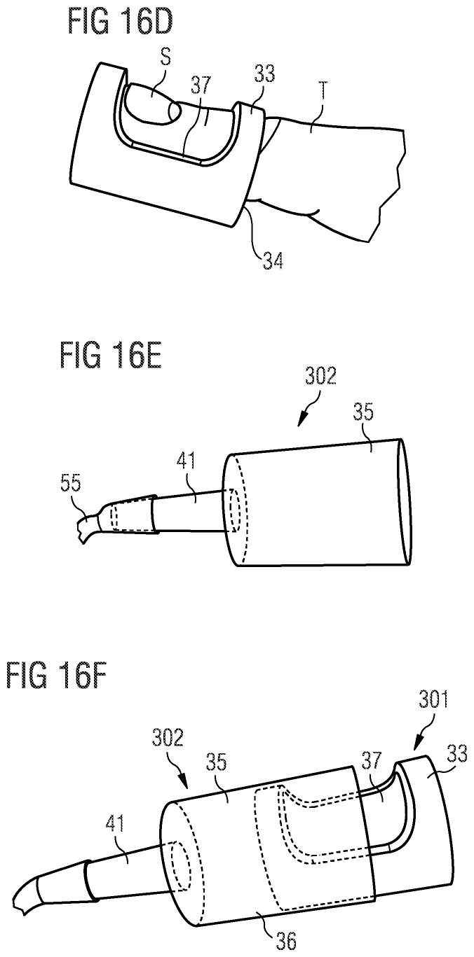

FIG. 12 shows a further embodiment of the apparatus 100 which has a sealing element 30 that is formed by two parts 30a, 30b that are an upper sealing part and a lower sealing part. The parts 30a, 30b can be made of a soft and flexible material that adapts to the treatment object T and seal the closed volume 40 against the surrounding atmosphere. The parts 30a, 30b form the side part 31, the mounting part 32 and the opening 34 as indicated in FIG. 12. For instance, at least the reception region 36 or the complete parts 30a, 30b can be at least partly formed by a memory foam or a silicone. The parts 30a, 30b can be pressed together due to a reduced gas pressure in the closed volume 40, which can be connected to a pump via a channel (not shown) in one of the parts 30a, 30b.

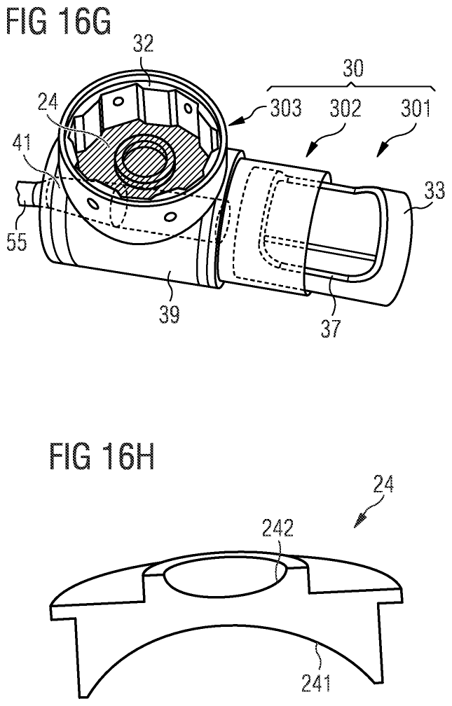

FIG. 13 shows a further embodiment of the apparatus 100, which has a sealing element 30 that is designed in such a way that its geometry is individually adapted to the treatment object T. The production of the individual geometry of the sealing element 30 can be carried out for instance via a three-dimensional measurement and a subsequent production by means of 3D printing. Alternatively, the geometry of the sealing element 30 can be adapted by a molding process, i.e., by molding the material for the sealing element 30 to the treatment object T. The molding process can for example be carried out by the formation of a negative mold with a rapidly hardening casting material and subsequently by molding a positive mold as geometry of the sealing element 30.

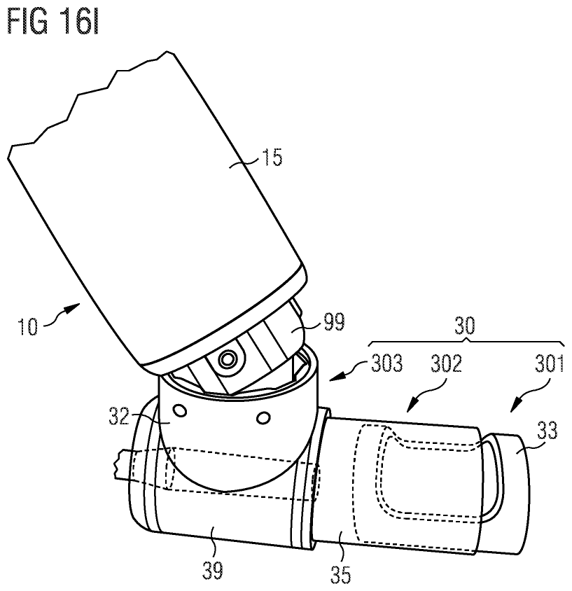

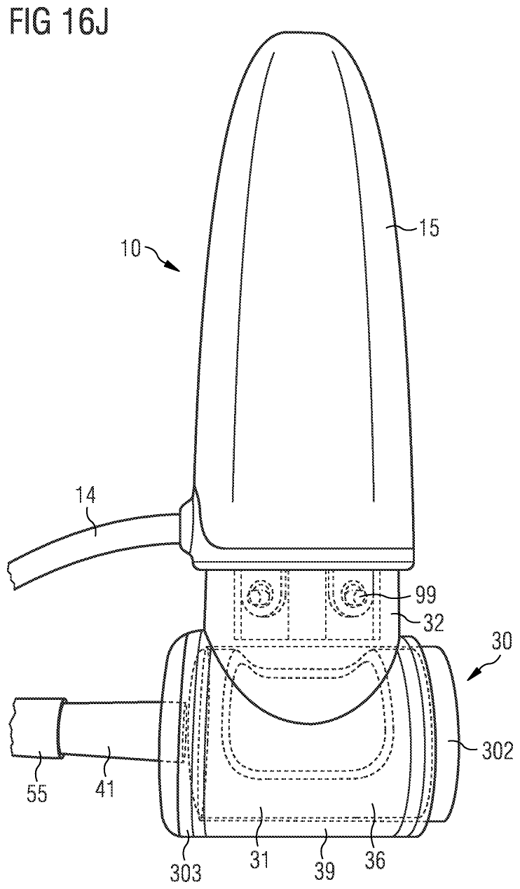

FIGS. 14A to 15B show further embodiments of the apparatus 100, which, in contrast to the foregoing embodiments, comprise a plasma-generating element 10 with a remote electrode 71 that is located in the cavity 35 of the sealing element 30 and is used to generate the plasma in the closed volume. Although the sealing element 30 is shown in FIGS. 14A to 15B as a dielectric treatment chamber with a tube-like shape, the plasma-generating element 10 with the remote electrode 71 can be combined with any of the sealing elements 30 of the foregoing embodiments.

The remote electrode 71 is connected to a high-voltage generator, which, in the shown embodiments, is embodied as piezoelectric transformer 1 in a housing 15 with a cap 20 as explained above. The output-side end region in the second region of the piezoelectric transformer 1 is connected to the remote electrode 71 via a conductor element 72 that can for instance be a conductive wire as indicated in FIGS. 14A and 14B or a conductive rod as indicated in FIGS. 15A and 15B. Accordingly, the conductor element 72, by means of which the sealing element 30 is attached to the housing 15 in the shown embodiments, in general forms a galvanic connection between the high-voltage generator and the remote electrode 71.

The output-side end region in the second region of the piezoelectric transformer 1 can be directly connected to the conductor element 72 or, as shown in FIGS. 14A to 15B, can be indirectly connected to the conductor element 72 by means of a conductive electrode 73. In the embodiments of FIGS. 14A and 15A, the conductive electrode 73 is formed by the electrically conductive film 24 on the cap 20 as explained in connection with FIG. 11. Alternatively, as shown in FIGS. 14B and 15B, the conductive electrode 73 can be an integral part of the cap 20 and can be embodied as an electrode inset that completely reaches through the cap 20, thus allowing direct exposure of the conductive electrode 73 to the primary plasma. Hence, the only dielectric barrier present is the surface and/or wall of the sealing element 30. In some embodiments, the conductive electrode 73 may be electrically associated to the output end of the piezoelectric transformer 1 via a non-Galvanic connection. For example, the conductive electrode 73 and the output end of the piezoelectric transformer 1 may be separated by a small gap, for example smaller than the distance between the remote electrode 73 and the treatment object. In such a case the remote electrode may be supplied with high voltage from the piezoelectric transformer via plasma, i.e., via charge carriers that drift across the gap.

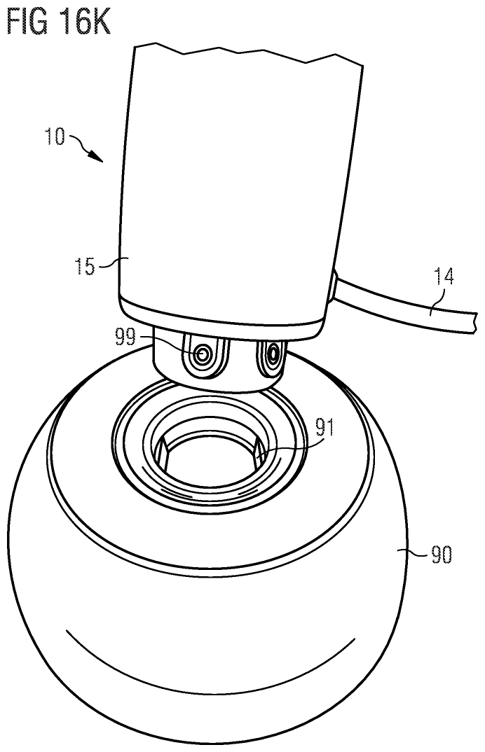

FIGS. 16A to 16K show a further embodiment of the apparatus 100, which is based on the principle as described for example in connection with FIGS. 8 to 11. Consequently, the apparatus 100 of the embodiment shown in FIGS. 16A to 16K comprises a sealing element 30 having a reception region 36 that is configured such that a part of the treatment object T, for instance a part of a finger or a toe, can be inserted into the reception region 36 which is a part of the cavity.

In contrast to the previous embodiments, the sealing element 30 is formed by several components 301, 302, 303 that are detachable and that can be assembled by inserting the components 301, 302, 303 into each other, thereby forming the sealing element 30. Furthermore, the apparatus 100 comprises a plasma-generating device 10 that can be provided separately from the sealing element 30 as described, for instance, in connection with FIG. 11.

FIG. 16A shows the apparatus 100 with the components 301, 302, 303 of the sealing element 30 disassembled and the sealing element 30 separated from the plasma-generating device 10. FIGS. 16B to 16K show various components of the apparatus 100 separately or partly assembled, respectively, in order to facilitate the understanding. The following description equally applies to the FIGS. 16A to 16K.

The plasma-generating device 10 can be embodied as explained in connection with any of the previous embodiments. Particularly preferably, the plasma-generating device 10 can be embodied as explained in connection with FIG. 2 or FIG. 3. Consequently, the plasma-generating device 10 can preferably comprise a piezoelectric transformer that is arranged in and supported by a housing 15. The output-side end region of the piezoelectric transformer can preferably be uncovered. The plasma-generating device 10 is electrically connected to an external electronics component 90 via wire connections 14, wherein control electronics can be situated inside the electronics component. The plasma-generating device 10 or the electronics component 90 can have a switch for switching on and off the plasma-generating device 10.

The electronics component 90 can additionally have a socket 91 in which a part of the plasma-generating device 10 fits that comprises a locking mechanism 99, so that the plasma-generating device 10 can be seated on the electronics component 90 when not used. The socket 91 can be formed as a counterpart for the locking mechanism 99. The locking mechanism 99 can comprise or be a spring lock, a snap-fit, a bayonet lock, a screw connection, a latch or similar and can provide secure means for easily and quickly mounting the plasma-generating device 10. In the shown embodiment, the locking mechanism 99 comprises several lock knobs.

The sealing element 30 comprises in the embodiment of FIGS. 16A to 16K three components 301, 302, 303, which are shown disassembled in FIG. 16A and assembled in FIG. 16J. Alternatively, the sealing element 30 can comprise two components or more than three components. It can also be possible that two of the shown three components are formed as a single component.

For example, the component 301 or both the components 301 and 302, which are described in detail in the following, can be disposable components that are used, for instance, only for one patient. Consequently, in particular those components that are in direct contact with the treatment object T can be disposed of after use for hygienic reasons.

In particular, the sealing element 30 comprises the first component 301 that is embodied as a sealing part having an opening 34 and a sealing lip 33. Furthermore, the sealing element 30 comprises the second component 302 that is embodied as a reception and cavity part into which the sealing part formed by the component 301 is inserted. In FIGS. 16B to 16D the first component 301 is shown in detail, wherein in FIG. 16D the position of the treatment object T is indicated when inserted into the component 301 through the opening 34. By way of example, the treatment object T is a finger. FIG. 16E shows the second component 302. FIG. 16F shows the first component 301 being half inserted into the second component 302.

For applications in connection with the treatment of a digit like a finger or a toe, the component 301 forming the sealing part can have a tube-like shape, as can be seen for instance in FIGS. 16B, 16C and 16D, preferably with inner dimensions that meet the dimensions of the treatment object T. For example, a collection of sealing parts can be provided which can be interchangeable and which have different inner dimensions, so that a suitable sealing part can be chosen depending on the size of the treatment object T to be treated. The first component 301 can preferably be formed by an elastic material like an elastomer. For example, the first component 301 can be made from rubber on the basis of caoutchouc or from a silicone elastomer. Accordingly, the first component 301 can form a cushioning element for bedding and supporting the treatment object T.

In the assembled sealing element 30, the first component 301, when chosen in the correct size with respect to the treatment object T, can be intended and embodied for sealing the cavity 35 from the outside environment. Alternatively, it can also be possible that the first component 301 is embodied without a sealing lip 34. In this case, an additional sealing component, for example a sealing ring, can form the sealing lip of the sealing element 30. The additional sealing component can be formed from an elastomer as a rubber on the basis of caoutchouc or a silicone elastomer and can also be a disposable component intended for a one-time-use that is replaced each time after treatment. The additional sealing component can be slid on the treatment object T in a first step. Afterwards, the first component 301 and the further components of the sealing element 30 can be arranged on the treatment object T. By pressing the first component 301 against the additional sealing component, combined with the suction of a vacuum pump as explained below, the closed volume can be sealed and a desired lower pressure or even vacuum can be created around the surface S to be treated. The sealing can be further improved by the additional sealing component and/or the first component 301 or any of the other components of the sealing element 30 having appropriate fittings relative to each other such as a tongue-and-groove system or similar.

Additionally, according to a preferred embodiment it is also possible that the sealing element 30 has no sealing lip and that during operation of the apparatus 100 gas from the surrounding atmosphere can be sucked or drawn into the cavity 35 and thus into the closed volume through the opening 33 as explained in further detail below.

In addition to the opening 34 through which the treatment object T can be inserted and which can, depending on the intended application, be formed with or without the sealing lip 33, the first component 301 has a treatment opening 37, so that the surface S of the treatment object T is accessible and not covered by the material of the first component.

The components 301 and 302 can substantially form the side part 31 forming the cavity 35 with the closed volume and, thus, the reception region 36 as indicated for example in FIG. 16J. The second component 302 can for example be cup-shaped with inner dimensions that correspond to the outer dimensions of the first component 301, so that, in case the first component 301 has the sealing lip 34, the sealing lip 33 can seal the closed volume when the treatment object T is inserted into the sealing element 30. As can be seen in FIG. 16F, the first component 301 can fit into the second component 302 in a piston-like manner.

The second component 302 can be made of a dielectric material, for example a plastic material. In particular, the second component 302 can form a dielectric barrier, so that outside the cavity 35 a primary plasma can be generated during operation of the apparatus, while inside the cavity 35 and, thus, inside the closed volume, when the treatment object T is inserted, a secondary plasma can be generated as described above.

Additionally, the second component 302 comprises a channel 41 through which a gas, for instance a reactive gas, can be inserted into the cavity 35 or through which gas can be pumped from the cavity 35, and thus from the closed volume when the treatment object T is inserted in the sealing element 30. In case the first component 301 has the shown sealing lip 34 or in case of an additional sealing component a lower pressure can be created in the closed volume. Reducing the pressure inside the closed volume can facilitate the generation of the plasma, which can lead to a reduction of possible unpleasant stimulations experienced by the patient. Furthermore, a reduced pressure can facilitate the distribution of the plasma inside the cavity 35, which can improve the therapeutic outcome. Alternatively, as described above, in case the sealing element 30 has no sealing lip during operation of the apparatus 100 gas from the surrounding atmosphere can be sucked or drawn into the closed volume through the opening 33 and can be removed from the closed volume through the channel 41. Accordingly, there can be a gas flow from the surrounding atmosphere through the closed volume for example to a pump and/or filter as described in the following. In case of such gas flow the pressure inside the closed volume can correspond, at least substantially, to the atmospheric pressure, i.e., the pressure of the surrounding atmosphere.

The channel 41 can extend from a part forming the cavity 35 as shown in FIG. 16E. In particular, the second component 302 can be formed similar to the front part of a plastic syringe. A tube 55 can be connected to the channel 41. For example, the electronics component 90 can comprise a pump and/or a filter that is connected to the channel 41 and, thus, to the cavity 35 of the sealing element 30 via the tube 55. The side of the first component 301 opposite the opening 34 can be formed as a closed bottom with venting holes 38, for example in the form of slits, as shown in FIGS. 16B and 16C. Through the venting holes 38 the gas transfer can be facilitated, so that a gas flow and/or gas distribution around the region of the treatment surface S can be facilitated.

Furthermore, an additional filter component can be arranged between the cavity 35 and the electronics component 90, wherein the additional filter component can preferably be intended and embodied for protecting the electronics component 90 and/or the environment from a microbiological contamination or another contamination caused by the treatment and/or treatment object T inside the cavity 35. The additional filter component can, for instance, also form a protection for the electronics component 90 against particles, for example skin flakes stemming from the treatment object T.

For example, the additional filter component can be arranged between the second component 302 and the tube 55. In particular, the additional filter component can be connected to the channel 41 and the tube 55 can be connected to a similar channel that is a part of the additional filter component. For a secure connection between the second component 302 and the additional filter component and/or between the additional filter component and the tube 55 or, in case no additional filter component is present, between the second component 302 and the tube 55, said components can be connected to each other by means of a connection technique as, for example, a standard Luer taper connection like a Luer slip or, preferably, a Luer lock. It can also be possible that the additional filter component can be arranged between the tube 55 and an additional tube leading from the additional filter component to the electronics component 90. Moreover, the additional filter component can be arranged between the tube 55 and the electronics component 90 and, thus, directly at the electronics component 90. Preferably, the additional filter component is arranged as close to the cavity 35 as possible.

The additional filter component can comprise or be a depth filter or a membrane filter. Preferably, the additional filter component can be a sterile filter which is typically used for filtering gases and/or liquids in medical and/or microbiological applications. Particularly preferably, the additional filter component is a syringe attachment filter embodied as a membrane filter. The pore size of the additional filter component can be preferably between 0.1 μm and 10 μm. The additional filter component can comprise, for instance, PTFE (polytetrafluoroethylene), nylon and/or cellulose as filter material(s). The additional filter component can be a disposable component intended for example for a one-time-use or a several-time-use, so that the additional filter component is replaced each time after one or several treatments.

Furthermore, the sealing element 30 comprises the third component 303 comprising the mounting part 32 for mounting the plasma-generating device 10 and a tube-like part 39 for accommodating the first and second components 301, 302. Accordingly, the third component is embodied as a coupling component coupling the first and second components 301, 302 to the plasma-generating device 10. An electrically conductive layer 24, for instance a metal film or layer, as described in connection with FIG. 10, can be situated in the mounting part 32. The conductive layer 24 can have a certain predefined distance to the piezoelectric transformer of the plasma-generating device 10 and furthermore can have direct access into the tube-like part 39, so that the conductive layer 24 can act as electrode for the plasma generation inside the second component 302 through the electrically insulating material of the second component 302 acting as a dielectric barrier as explained above. In particular, in a preferred embodiment the second component 302 can form the only dielectric barrier between the plasma-generating device 10 and the treatment object T. Accordingly, the conductive layer 24 is directly accessible from the mounting part 32 and the tube-like part 39 and can be formed as an electrode inset in the third component 303.

In FIG. 16G the components 301, 302 and 303 are shown in a half-assembled state, wherein the first component 301 is half inserted in the second component 302 and the second component 302 is half inserted in the third component. FIG. 16I shows the first component 301 nearly fully inserted in the second component 302, but the second component 302 nearly not inserted in the tube-like part 39 of the third component 303. Furthermore, FIG. 16I additionally shows the plasma-generating device 10 half inserted in the mounting part 32. For detachably locking the plasma-generating device 10 to the sealing element 30 and, in particular, in the mounting part 32, the mounting part 32 comprises counterparts for the locking mechanism 99 of the plasma-generating device. FIG. 16J shows the sealing element 30 fully assembled with the mounted and locked plasma-generating device 10.

Preferably, at least the second and third component 302, 303 comprise or are at least partly made from transparent materials, for instance transparent plastics, so that the surface S of the treatment object T is visible through the components 302, 303 and the treatment opening 37 of the first component 301. Preferably, the third component 303 is movable at least with respect to the second component 302. In particular, the part 39 of the third component 303 as well as the second components 302 can have a substantially cylindrical shape so that the mounting part 32 and, thus, the plasma-generating device 10 can be rotated around the cylinder axis and moved along the cylinder axis relative to the first and second component 301, 302. Due to the movement a larger area of the surface S is accessible. For example in case of a finger or toe it can be ensured by such movability that the complete nail can be treated.

It can also be possible that the conductive layer 24 has a shape on the side facing the treatment object T that is adapted to the geometrical form and/or size of the surface S to be treated. For instance, the conductive layer 24 can be formed as a so called saddle electrode and can have a concavely shaped surface 241 on the side facing the treatment object T as indicated in FIG. 16H showing a sectional view of the conductive layer 24. Alternatively, the conductive layer 24 can be formed as a so called bump electrode having a convexly shaped surface on the side facing the treatment object. It can also be possible that the second sealing component 302 is not completely cylindrical but has a protuberance into which the conductive layer 24 reaches.

In a preferred embodiment at least some of the components of the sealing element 30, for example the first component 301 and/or the second component 302 and/or at least the conductive layer 24 of the third component 303 are adapted, particularly preferably accurately fitting, to the treatment object T and the surface S to be treated. By such means it can be possible to concentrate the plasma substantially to that region where it is needed, for instance only to a region with a fungus infection of a nail so that the plasma is not applied to the complete nail and an efficient treatment can be ensured. Furthermore, by adapting the side of the conductive layer 24 facing the treatment object and/or the second component 302 to the geometrical form of the surface to be treated a substantially equal distance of the conductive layer 24 and/or of the dielectric barrier formed by the second component 302 to the surface S can be achieved so that the effect of the plasma can be substantially homogeneous over the whole surface S.

Furthermore, for increasing the coupling between the plasma-generating device 10 and the conductive layer 24 and, as a consequence, for reducing the energy dissipation of a plasma arcing between the plasma-generating device 10 and the conductive layer 24, the conductive layer 24 can have projection 242 on the side facing the plasma-generating device as shown in FIG. 16H. The projection 242 can have a convexly formed surface facing the plasma-generating element of the plasma-generating device 10, i.e., a piezoelectric transformer as explained above. In particular, the projections 242 is formed to at least partly surround the output-side end region of the piezoelectric transformer and to reduce the gap to the output-side end region so that the surface of the projection is as close as possible to the output-side end region of the piezoelectric transformer in order to effectively transfer energy from the piezoelectric transformer to the conductive layer 24 and to reduce energy dissipation in the plasma arcing as far as possible.

Accordingly, the sealing element 30 has a first gap between the plasma-generating element and the conductive layer 24. A second gap can be situated between the surface 241 of the conductive layer 24 and the second component 302 forming a dielectric barrier. A third gap can be situated between the second element 302 and the surface S to be treated. For the treatment of the surface S of the treatment object T the secondary plasma in the third gap is desired, whereas primary plasmas in second gaps are substantially undesired. As explained above, by adapting the shapes of the components of the sealing element 30 to each other and to the surface S of the treatment object T an efficient energy transfer can be achieved from the plasma-generating device 10 to the surface S, wherein the generation of undesired primary plasmas in the first and second gap can be reduced or even suppressed.

Alternatively or additionally to the features described in connection with the figures, the embodiments shown in the figures can comprise further features described in the general part of the description. Moreover, features and embodiments of the figures can be combined with each other, even if such combination is not explicitly described.

The invention is not restricted by the description on the basis of the exemplary embodiments. Rather, the invention encompasses any new feature and also any combination of features, which in particular comprises any combination of features in the patent claims, even if this feature or this combination itself is not explicitly specified in the patent claims or exemplary embodiments.

### REFERENCE NUMERALS

1 piezoelectric transformer
2 first region
3 second region
4 electrode
5 piezoelectric material
6, 7 side surface
8 electrode
9 piezoelectric material
10 plasma-generating element
11 potting compound
12, 13 end region
14 wire connection
15 housing
15a, 15b housing section
16a, 16b bend
17 cover
18 support element
19 displacement protection element
20 cap
21, 22 plasma
23 gas space
24 electrically conductive layer
30 sealing element
30a, 30b part of sealing element
31 side part
31a, 31b part of the side part
32 mounting part
33 sealing lip
34 opening
35 cavity
36 reception region
37 treatment opening
38 venting hole
39 part
40 closed volume
41 channel
51 pump
52 filter
53 gas tank
54 valve
55 tube
60 protection cover
71 remote electrode
72 conductor element
73 conductive electrode
90 electronics component
91 socket
99 locking mechanism
100 apparatus
241 surface
242 projection
301, 302, 303 component
S surface
T treatment object

1. An apparatus for generating a plasma above a treatment object, the apparatus comprising:
a plasma-generating element, wherein, during operation, the plasma is generated by the plasma generating element, and
a sealing element attached to the plasma-generating element,
wherein the sealing element has a cavity and is configured to form a closed volume with a part of the treatment object, so that at least a part of the cavity is part of the closed volume and, during operation of the apparatus, the plasma is generated in the closed volume,
wherein the sealing element comprises a reception region that is configured so that a part of the treatment object can be inserted into the reception region,
wherein the sealing element comprises detachable components, and the sealing element comprises at least a first component and a second component, wherein the treatment object can be inserted in the first component and the first component is inserted in the second component, and/or the sealing element comprises at least a second component and a third component, wherein the second component is inserted in the third component, and the third component is movable with respect to the second component.

2. The apparatus according to claim 1, wherein the sealing element has a sealing lip surrounding the opening.

3. (canceled)

4. The apparatus according to claim 1, wherein the reception region is at least partly formed by a tube or a memory foam.

5. The apparatus according to claim 1, wherein a housing of the plasma-generating element and/or the sealing element has at least one channel reaching into the cavity of the sealing element.

6. The apparatus according to claim 5, wherein the apparatus further comprises a pump and/or a filter and the at least one channel is connected to the pump and/or the filter.

7-8. (canceled)

9. The apparatus according to claim 1, wherein the first component comprises a treatment opening so that, when the treatment object is inserted in the first component, a surface of the treatment object is not covered by material of the first component.

10. (canceled)

11. The apparatus according to claim 1, wherein the third component comprises a mounting part for mounting the plasma-generating device and a tube-like part for accommodating the second component.

12. The apparatus according to claim 1, wherein one of the components of the sealing element forms a dielectric barrier being the only dielectric barrier between the plasma-generating device and the treatment object.

13. The apparatus according to claim 1, wherein the plasma-generating device has a locking mechanism for detachably locking the plasma generating device to the sealing element.

14. The apparatus according to claim 1, wherein the plasma-generating element comprises
a piezoelectric transformer, the piezoelectric transformer comprising a first region and a second region, wherein, during operation of the apparatus, the plasma is generated in the vicinity of an end region in the second region of the piezoelectric transformer, and
a housing enclosing at least a part of the first region of the piezoelectric transformer.

15-16. (canceled)

17. The apparatus according to claim 14, wherein the sealing element comprises an electrically conductive layer arranged between the piezoelectric transformer and the cavity.

18. The apparatus according to claim 17, wherein the electrically conductive layer comprises a projection facing the piezoelectric transformer.

19. Method for performing a plasma treatment to a treatment object using the apparatus according to claim 1,
wherein the apparatus is placed on the treatment object so that a part of the treatment object is inserted into a part of the cavity of the sealing element, so that the closed volume is formed by the sealing element and by the treatment object,
wherein the apparatus is operated to generate a plasma above the surface of the treatment object.

20. (canceled)

21. A device for treating a treatment object with plasma, the device comprising:
a piezoelectric transformer having an input region and an output region, the piezoelectric transformer being configured to transform a low voltage at the input region to a high voltage at the output region,
a plasma-applying electrode, electrically associated with the output region of the piezoelectric transformer via an elongated conductor, and
a dielectric layer positioned between the plasma-applying electrode and the treatment object,
wherein the plasma-applying electrode is configured to apply plasma adjacently to a surface of the treatment object in a dielectric barrier discharge mode.

22. The device of claim 21, wherein the plasma applying electrode is electrically associated with the piezoelectric transformer via a non-Galvanic connection.

23. The device of claim 21, wherein the conductor element is directly connected to the output region.

24. The device of claim 21, wherein the conductor element is indirectly connected to the output region via a conductive electrode.

25. The device of claim 21, further comprising a dielectric barrier between the piezoelectric transformer and the plasma applying electrode.

26. The device of claim 21, wherein the plasma applying electrode and/or the dielectric layer are adapted to the geometrical form of the surface of the treatment object, thereby establishing a substantially equal distance to the surface.

27. The device of claim 21,
wherein the dielectric layer is part of a sealing element having a cavity, configured to form a closed volume with a part of the treatment object, so that at least a part of the cavity is part of the closed volume, and, during operation of the device, the plasma is generated in the closed volume, and
wherein the sealing element comprises a reception region configured so that a part of the treatment object can be inserted into the reception region.
